SEQUENCE LISTING (6 sequences)

SEQ ID NO: 1 - DNA, 20 bp, Artificial Sequence
  Ndufb5 forward
tcccagaaggctacatccct

SEQ ID NO: 2 - DNA, 20 bp, Artificial Sequence
  Ndufb5 reverse
attccgggcgatccatcttg

SEQ ID NO: 3 - DNA, 20 bp, Artificial Sequence
  Uqcrb forward
tcaagcaagtggctggatgg

SEQ ID NO: 4 - DNA, 20 bp, Artificial Sequence
  Uqcrb reverse
tcaggtccagggctctctta

SEQ ID NO: 5 - DNA, 20 bp, Artificial Sequence
  Cycs forward
aatctccacggtctgttcgg

SEQ ID NO: 6 - DNA, 20 bp, Artificial Sequence
  Cycs reverse
ggtctgccctttctcccttc

(19) United States
(12) Patent Application Publication
(10) Pub. No.: US 2018/0256519 A1
(43) Pub. Date: Sep. 13, 2018

(54) COMPOSITION FOR PREVENTING OR TREATING MITOCHONDRIAL DISEASES CAUSED BY IMMUNOSUPPRESSANTS, AND IMMUNE DISEASES, CONTAINING METFORMIN

(71) Applicant: THE CATHOLIC UNIVERSITY OF KOREA INDUSTRY-ACADEMIC COOPERATION FOUNDATION, Seoul (KR)

(72) Inventors: Mi La Cho, Seoul (KR); Chul Woo Yang, Seoul (KR); Sung Hwan Park, Seoul (KR); Seon Yeong Lee, Seoul (KR); Min Jung Park, Seoul (KR); Joo Yeon Jhun, Seoul (KR); Sun Woo Lim, Seoul (KR); Byung Ha Chung, Seoul (KR); Eun Kyung Kim, Seoul (KR); Jae Kyung Kim, Seoul (KR); Hyun-Sik Na, Seoul (KR); Se-Young Kim, Seoul (KR); Eun Jung Lee, Gyeonggi-do (KR); Hyeon-Beom Seo, Seoul (KR)

(21) Appl. No.: 15/903,675

(22) Filed: Feb. 23, 2018

Related U.S. Application Data
(63) Continuation of application No. PCT/KR2016/009376, filed on Aug. 24, 2016.

(30) Foreign Application Priority Data
Aug. 24, 2015 (KR) 10-2015-0118934

(51) Int. Cl. A61K 31/155; A61P 37/06

(52) U.S. Cl. CPC A61K 31/155; A61K 31/436; A61K 2300/00; A61P 37/06

(57) ABSTRACT

The present invention relates to a composition for preventing or treating mitochondrial diseases caused by immunosuppressants, and immune diseases, containing metformin and, more specifically, to a composition for treating mitochondrial diseases caused by immunosuppressants, containing metformin; a pharmaceutical composition for preventing or treating immune diseases, containing, as active ingredients, metformin and an immunosuppressant, which is a target of rapamycin inhibitor (mTOR inhibitor); and a pharmaceutical composite formulation for preventing or treating immune diseases, containing, as ingredients, metformin and a mammalian target of rapamycin inhibitor, wherein the metformin and mammalian target of rapamycin inhibitor are administered simultaneously or separately, or administered in a predetermined sequence. The composition effectively alleviates mitochondrial dysfunction, occurring as a side effect of conventional immunosuppressants, while having a more improved immunosuppressive therapeutic effect, thereby being usable in prevention and treatment of transplant rejection, autoimmune diseases, inflammatory diseases, and the like, all of which require immunosuppression.

### TECHNICAL FIELD

This application claims priority from and the benefit of Korean Patent Application No. 10-2015-0118934 filed on 24 Aug. 2015, which is hereby incorporated in its entirety by reference.

The present invention relates to a composition comprising metformin for preventing or treating an immunosuppressant-induced mitochondrial disease and an immune disease and, more specifically, to a composition comprising metformin for preventing or treating an immunosuppressant-induced mitochondrial disease, to a pharmaceutical composition comprising, as active ingredients, metformin and a mammalian target of rapamycin inhibitor (mTOR inhibitor), which is an immunosuppressant, for preventing or treating an immune disease, and to a pharmaceutical complex preparation for preventing or treating an immune disease, the pharmaceutical complex preparation being characterized by containing, as ingredients, metformin and a mammalian target of rapamycin inhibitor, which are administered simultaneously, individually, or in a predetermined order.

### BACKGROUND ART

Immunosuppressants are drugs that block or reduce the humoral immune response or cell-mediated immune response of making antibodies to antigens, and have been mainly used to treat immune rejection response after organ transplantation or graft-versus-host disease after bone marrow transplantation. In addition, immunosuppressants are significantly used for the long-term treatment of symptoms of autoimmune diseases such as lupus and rheumatoid arthritis, hyperimmune responses such as allergy and atopic disease, and inflammatory diseases.

The immunosuppressants that are currently used are classified, according to the mechanism of action, into corticosteroids, antimetabolites, calcineurin inhibitors, mammalian targets of rapamycin inhibitors, antibodies, and the like, which exhibit an immunosuppressive effect by blocking the proliferation or activation of T cells in the immune system at different stages, respectively (Dalal, P. et al. Int. J. Nephrol. Renovasc. Dis. 3:107-115 (2010)). T cells, which are the main target of immunosuppressants, are formed in the thymus of the human body, and are mainly differentiated into type 1 auxiliary cells (Th1) involved in cell-mediated immunity or type 2 auxiliary cells (Th2) involved in humoral immunity. It is known that two T cell groups hold each other in check so that the T cell groups are not excessively activated, and then the breakage of balance therebetween induces an abnormal response, such as autoimmunity or hypersensitivity.

Above these, new types of T cells, such as immunoregulatory T cells (Treg) or Th17 cells, which are capable of regulating immune responses, are known. Treg can regulate Th1 cell activity, and suppresses functions of abnormally activated immune cells and regulates inflammatory responses. In contrast, Th17 cells secrete IL-17, and maximize signals of inflammatory responses to accelerate the disease progression. Recently, Treg or Th17 has been highlighted as a new target for immune disease drugs, so various immunoregulatory drugs have been studied (Wood, K. J. et al. Nat. Rev. Immunol. 12(6):417-430 (2012), Miossec, P. et al. Nat. Rev. Drug Discov. 11(10):763-776 (2012), Noack, M. et al. Autoimmun. Rev. 13(6):668-677 (2014)).

Existing immunosuppressants that nonspecifically suppress T cells are generally accompanied by side effects, such as cytotoxicity, infections due to immunodeficiency, diabetes, tremor, headache, diarrhea, hypertension, nausea, and renal dysfunction, and thus have difficulty in sustaining treatment effects over a long period of time. In order to reduce serious side effects of immunosuppressants and increase immunosuppressive treatment effects thereof, in particular, the field of organ transplantation, the methods of co-administering immunosuppressants with different mechanisms of action or administering one type of drug for a certain period of time and then replacing the drug with another type of drug have been attempted, but optimized combinations or therapies for co-administration of immunosuppressants have not yet been established.

Therefore, new immunosuppressive or immunoregulatory therapies capable of reducing side effects of existing immunosuppressants and increasing the treatment effects thereof and new immunosuppressant candidates having excellent safety and few side effects need to be urgently developed.

### DETAILED DESCRIPTION OF THE INVENTION

### Technical Problem

The present inventors, while conducting research about new immunoregulators capable of having few side effects and exhibiting sustained treatment effects, confirmed that the co-administration of metformin and an immunosuppressant based on a mammalian target of rapamycin (mTOR) inhibitor produces synergistic effects in the immunoregulation or immunosuppression, such as the inhibition of inflammatory cytokine secretion and the activation of Treg cells, and especially first discovered that metformin has an effect of improving mitochondrial functions impaired by side effects of existing immunosuppressants, and thus the present inventors completed the present invention.

Therefore, an aspect of the present invention is to provide a pharmaceutical composition for treating an immunosuppressant-induced mitochondrial disease, the composition comprising, as an active ingredient, metformin or a pharmaceutically acceptable salt thereof.

Another aspect of the present invention is to provide a pharmaceutical composition for treating an immune disease, the composition comprising, as active ingredients, an mTOR inhibitor and metformin or a pharmaceutically acceptable salt thereof.

Still another aspect of the present invention is to provide a pharmaceutical complex preparation for treating an immune disease, the pharmaceutical complex preparation being characterized in that:

(a) the pharmaceutical complex preparation contains an mTOR inhibitor and metformin or a pharmaceutically acceptable salt thereof at a weight ratio of 1:500 to 1:200,000; and

(b) the mTOR inhibitor and metformin or the pharmaceutically acceptable salt thereof are administered simultaneously, individually, or in a predetermined order.

Another aspect of the present invention is to provide a use of metformin or a pharmaceutically acceptable salt thereof for preparing an agent for treating an immunosuppressant-induced mitochondrial disease.

Another aspect of the present invention is to provide a method for treating an immunosuppressant-induced mitochondrial disease, the method being characterized by administering an effective amount of a composition to a subject in need thereof, the composition comprising, as an active ingredient, metformin or a pharmaceutically acceptable salt thereof.

Another aspect of the present invention is to provide a use of an mTOR inhibitor and metformin or a pharmaceutically acceptable salt thereof for preparing an agent for treating an immune disease.

Another aspect of the present invention is to provide a method for treating an immune disease, the method being characterized by administering an effective amount of a composition to a subject in need thereof, the composition comprising, as active ingredients, an mTOR inhibitor and metformin or a pharmaceutically acceptable salt thereof.

### Technical Solution

In accordance with an aspect of the present invention, there is provided a pharmaceutical composition for treating an immunosuppressant-induced mitochondrial disease, the composition comprising, as an active ingredient, metformin or a pharmaceutically acceptable salt thereof.

In addition, the present invention provides a composition consisting of metformin or a pharmaceutically acceptable salt thereof.

In addition, the present invention provides a composition essentially consisting of metformin or a pharmaceutically acceptable salt thereof.

In accordance with another aspect of the present invention, there is provided a pharmaceutical composition for treating an immune disease, the composition comprising, as active ingredients, an mTOR inhibitor and metformin or a pharmaceutically acceptable salt thereof.

In addition, the present invention provides a composition consisting of an mTOR inhibitor and metformin or a pharmaceutically acceptable salt thereof.

In addition, the present invention provides a composition essentially consisting of an mTOR inhibitor and metformin or a pharmaceutically acceptable salt thereof.

In accordance with still another aspect of the present invention, there is provided a pharmaceutical complex preparation for treating an immune disease, the pharmaceutical complex preparation being characterized in that: (a) the pharmaceutical complex preparation contains an mTOR inhibitor and metformin or a pharmaceutically acceptable salt thereof at a weight ratio of 1:500 to 1:200,000; and

(b) the mTOR inhibitor and metformin or the pharmaceutically acceptable salt thereof are administered simultaneously, individually, or in a predetermined order.

In accordance with an aspect of the present invention, there is provided a use of metformin or a pharmaceutically acceptable salt thereof for preparing an agent for treating an immunosuppressant-induced mitochondrial disease.

In accordance with another aspect of the present invention, there is provided a method for treating an immunosuppressant-induced mitochondrial disease, the method being characterized by administering an effective amount of a composition to a subject in need thereof, the composition comprising, as an active ingredient, metformin or a pharmaceutically acceptable salt thereof.

In accordance with another aspect of the present invention, there is provided a composition consisting of metformin or a pharmaceutically acceptable salt thereof.

In accordance with another aspect of the present invention, there is provided a composition essentially consisting of metformin or a pharmaceutically acceptable salt thereof.

In accordance with another aspect of the present invention, there is provided a use of an mTOR inhibitor and metformin or a pharmaceutically acceptable salt thereof for preparing an agent for treating an immune disease.

In accordance with another aspect of the present invention, there is provided a method for treating an immune disease, the method being characterized by administering an effective amount of a composition to a subject in need thereof, the composition comprising, as active ingredients, an mTOR inhibitor and metformin or a pharmaceutically acceptable salt thereof.

In accordance with another aspect of the present invention, there is provided a composition consisting of an mTOR inhibitor and metformin or a pharmaceutically acceptable salt thereof.

In accordance with another aspect of the present invention, there is provided a composition essentially consisting of an mTOR inhibitor and metformin or a pharmaceutically acceptable salt thereof.

Hereinafter, the present invention will be described in detail.

The present invention provides a pharmaceutical composition for treating an immunosuppressant-induced mitochondrial disease, the composition comprising, as an active ingredient, metformin or a pharmaceutically acceptable salt thereof.

The term “metformin” refers to a biguanide-based compound having a structure of chemical formula (C₄H₁₁N₅) below and a molecular weight of 129.16 Da. Metformin has long been used as an antidiabetic agent, especially for the treatment of type 2 diabetes. Metformin is marketed under the trade mark Glucophage, and various generic drugs thereof are marketed.

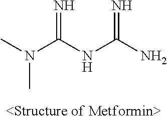

The term “immunosuppressant” refers to a drug that suppresses activity of the immune system. The immunosuppressant in the present invention may be preferably a mammalian target of rapamycin (mTOR) inhibitor, and most preferably rapamycin or a derivative thereof.

The term “mammalian target of rapamycin inhibitor (mTOR inhibitor)” refers to an agent that inhibits or suppresses the activity of a mammalian target of rapamycin. The “mTOR (mammalian target of rapamycin” or “mechanistic target of rapamycin)” is serine/threonine kinase belonging to the phosphoinositide 3-kinase (PI3K)-related kinase family and has a molecular weight of 289 kDa, and is a key regulation factor in the metabolism, growth, proliferation, and survival of cells. The mTOR is also known as FRAP, FRAP1, FRAP2, RAFT1, RAPT1, and the like. The mTOR functions by binding to another protein to form mTOR Complex 1 (mTORC1) or mTOR Complex 2 (mTORC2). The mTOR is involved in tumorgenesis, angiogenesis, insulin resistance, adipogenesis, T-lymphocyte activation, and the like, and is abnormally regulated in various diseases including, particularly, tumorigenic diseases, and thus the mTOR inhibitor is used as a medicine for these diseases.

The term “rapamycin” refers to the macrolide lactone compound having a structure of the chemical formula (C₅₁H₇₉NO₁₃) below and a molecular weight of 914.2 Da, and is also called sirolimus. Rapamycin binds to intracytoplasmic FK-binding protein 12 (FBP12) to form a complex and suppresses mTOR activity. In the immune system, rapamycin inhibits signaling associated with IL-2 and other cytokine receptors and prevents the proliferation and activation of T cells and B cells in the immune system. Due to such an immunosuppressive effect, rapamycin has been widely used as an immunosuppressant for organ transplantation or autoimmune diseases. Especially, rapamycin is utilized in the field of kidney transplantation since rapamycin has lower toxicity to the kidney compared with an immunosuppressant that inhibits calcineurin, such as cyclosporin or tacrolimus. Nevertheless, rapamycin exhibits toxicity, such as gastric mucosal ulceration, weight loss, diarrhea, and thrombocytopenia, in animal models, and has side effects, such as gastrointestinal disorders, hyperlipidemia, lung toxicity, and possibility of cancer occurrence due to immunosuppression, and thus the widespread use of rapamycin is limited. Rapamycin as an immunosuppressant is typically marketed as Rapamune from Pfizer Inc., or the like. As a patent of rapamycin associated with the inhibition of the rejection of organ transplantation expires, development strategies in association with an administration method for improving the immunosuppressive efficacy of rapamycin and compensating side effects thereof and a co-administration of rapamycin with another drug have been attempted.

Rapalogs, which are rapamycin derivatives, include temsirolimus, everolimus, deforolimus, and the like. Temsirolinms (chemical formula: C₅₆H₈₇NO₁₆, molecular weight 1030.3 Da) is an mTOR-specific inhibitor, and also known as Torisel or CCI-779. Everolimus (chemical formula: C₅₃H₈₃NO₁₄, molecular weight: 958.2 Da) is a 40-O-(2-hydroxyethyl) derivative of rapamycin, and known as RAD001 or the trademark Zortress, Certican, or Afinitor, and acts similar to rapamycin. Everolimus is currently being used as an immunosuppressant for organ transplantation. Deforolimus (chemical formula: C₅₃H₈₄NO₁₄P, molecular weight: 990.22 Da) is an mTOR inhibitor and also known as ridaforolimus, AP23573, MK-8669, or the like.

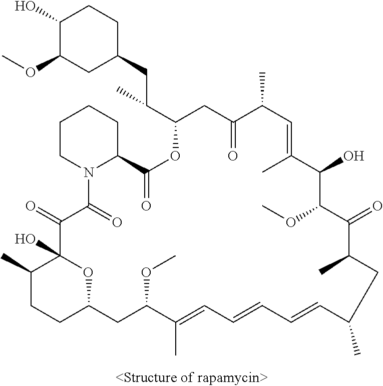 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

The term “mitochondrial disease” refers to a disease caused by mitochondrial dysfunction, and includes diseases caused by: the dysfunction due to the swelling by the mitochondrial membrane potential abnormality and due to the oxidative stress by active oxygen species or free radicals; the dysfunction due to genetic factors, such as genetic mutation associated with mitochondrial DNA or nuclear mitochondrial functions; and the defects of mitochondrial oxidative phosphorylation functions for energy production. Mitochondria are essential cell organelles that produce the cellular energy ATP, and the mitochondrial dysfunction inhibits the functions of all the cells containing mitochondria, other than red blood cells not containing mitochondria, and affects, especially, organs demanding high energy, such as muscles and the brain.

Examples of the disease that occurs directly due to mitochondrial dysfunction include: Leber's hereditary optic neuropathy; Leigh syndrome; neuropathy; ataxia; neuropathy, ataxia, retinitis pigmentosa, and ptosis (NARP); encephalomyelitis; myoclonic epilepsy and ragged red fibers (MERRF); mitochondrial myopathy, encephalomyopathy, lactic acidosis, stroke-like symptoms (MELAS); mitochondrial myopathy; Reye syndrome; Alper's disease; Friedrich's Ataxia; and the like. It has been recently known that mitochondrial functions are important in the induction and progression of a variety of other known diseases, for example, ischemic diseases, such as ischemic brain disease and ischemic heart disease, multiple sclerosis, polyneuropathies, migraine, depression, seizure, dementia, palsy, optic atrophy, optic neuropathy, glaucoma, retinitis pigmentosa (RP), cataract, hyperaldosteronism, hypoparathyroidism, myopathy, myatrophy, myoglobinuria, muscle tension inhibition, muscle pain, decreased exercise tolerance, tubulopathy, renal insufficiency, hepaticinsufficiency, hepatic dysfunction, hypertrophy, anaemia, neutropenia, thrombocytopenia, diarrhea, villous atrophy, multiple vomiting, dysphagia, constipation, sensorineural deafness, mental retardation, epilepsy, Alzheimer's disease, Parkinson's disease, Huntington's disease, and the like.

In particular, the dysfunction of mitochondria, which are essential for cellular energy metabolism, has been revealed to be also important in various types of energy and metabolic diseases, such as diabetes, obesity, and metabolic syndrome. It has been reported that diabetes mellitus and deafness (DAD) occurs directly due to point mutation at the 3243rd position of human mitochondrial DNA and the reduction in mitochondrial size and the deterioration of mitochondrial activity, such as the reductions in mitochondrial respiratory activity and electron transport system activity, due to the oxidative stress in the body, are highly correlated with the onset of diabetes.

The term “immunosuppressant-induced mitochondrial disease” refers to a disease due to the deterioration of mitochondrial activity caused by side effects of an immunosuppressant, and includes, for example, mitochondrial respiration disorder, impairment of mitochondrial membrane potential maintenance function, quantitative reduction of mitochondria, abnormal expression of mitochondrial function-related genes, and the like. Preferably, the immunosuppressant-induced mitochondrial disease may be caused by at least one mitochondrial dysfunction selected from mitochondrial respiration suppression, mitochondrial membrane potential reduction, and mitochondrial activity reduction. As described above, the immunosuppressant-induced mitochondrial dysfunction may be shown as, especially, a metabolic disorder, such as diabetes.

The present inventors first observed through cell experiments that rapamycin induced mitochondrial dysfunction and that the co-treatment with rapamycin and metformin improved mitochondrial dysfunction caused by rapamycin. In addition, the present inventors also confirmed that animals administered with rapamycin for a long period of time showed diabetic-like symptoms and that the co-administration of rapamycin and metformin could improve diabetic symptoms. Therefore, it can be seen that the composition comprising as an active ingredient metformin or a pharmaceutically acceptable salt thereof according to the present invention can be used to improve mitochondrial dysfunction caused by an mTOR inhibitor, such as rapamycin.

The effects of the co-administration of metformin and rapamycin on the improvement of mitochondrial functions were established by the present inventors as follows.

In an example of the present invention, rapamycin was observed to reduce the mitochondrial respiration as measured by the mitochondrial oxygen consumption rate in synovial cells, and especially, to remarkably reduce the increase in respiratory rate due to the treatment with FCCP as an uncoupling agent. The baseline rate of mitochondirial respiration was increased in the co-treatment with rapamycin and metformin compared with the treatment with rapamycin alone, and the co-administration of oligomycin as an ATP synthase inhibitor or FCCP, together with metformin, also increased the mitochondrial respiratory rate. That is, it can be seen that metformin improves the mitochondrial respiration disorder due to rapamycin.

In another example of the present invention, the amount of mitochondria stained with MitoTracker was significantly decreased in the synovial cells treated with rapamycin (1 nM) alone, but the amount of mitochondria in the co-treatment with rapamycin and metformin (200 nM or 1 mM) was maintained at the level as in a control group untreated with drugs. That is, it can be seen that metformin restores the quantitative reduction of mitochondria due to rapamycin.

In another example of the present invention, it was confirmed that the mitochondrial membrane potential observed through JC-1 staining was not maintained at the normal level in the synovial cells treated with rapamycin (1 nM) alone, but the mitochondrial membrane potential was maintained at the normal level in the co-treatment with rapamycin and metformin (200 nM or 1 mM). That is, it can be seen that metformin prevents an abnormal mitochondrial membrane potential.

In another example of the present invention, as a result of RT-PCR measurement of the expression levels of NADH dehydrogenase (ubiquinone) 1 beta subcomplex, 5, 16 kDa (Ndufb5), ubiquinol-cytochrome c reductase binding protein (Uqcrb), and cytochrome c (Cycs), which are associated with essential functions of mitochondria in NIH3T3 cells, the expression levels of these genes were observed to remarkably increase in the co-treatment with rapamycin and metformin (200 uM or 1 mM) rather than the treatment with rapamycin (1 mM) alone. The metformin promotes the expression of mitochondria-related genes, indicating that metformin has a possibility of improving other mitochondrial dysfunctions by increasing the expression of mitochondrial function related genes.

In another example of the present invention, as a result of subcutaneous injection of rapamycin (0.3 mg/kg) into rats for 6 weeks, the body weight decreased and the urine volume increased compared with a control group, and especially, the rats showed diabetic symptoms in the glucose tolerance and insulin resistance tests. It was confirmed that the rats co-administered with rapamycin and metformin from 3.5 weeks after rapamycin administration showed improved diabetic symptoms compared with a rapamycin administered alone group.

The above examples of the present invention show that the use of rapamycin as an immunosuppressant can suppress immune responses, such as inflammation, but is accompanied by side effects, such as mitochondrial function impairment. It can be seen that the mitochondrial dysfunction by rapamycin can be improved by simultaneous administration of rapamycin and metformin, separate administration of rapamycin and metformin during the administration period of rapamycin, or administration of metformin before the start of administration of rapamycin or after the end of the administration period of rapamycin.

In addition, the present invention provides a pharmaceutical composition for treating an immune disease, the composition comprising as active ingredients an mTOR inhibitor and metformin or a pharmaceutically acceptable salt thereof.

The mTOR inhibitor may be preferably rapamycin or a derivative thereof. The mTOR inhibitor, rapamycin, and derivative of rapamycin are as described above.

Recently, the present inventors have first discovered and reported that metformin has effects of regulating the balance of Treg/Th17 immune cells by inhibiting pathologic Th17 cells and inducing the differentiation of Treg cells regulating inflammation (Song, J. H. et al. Mediators Inflamm. 2014, Article ID 973986 (2014)). Therefore, the present inventors confirmed through experiments using immune cells that the co-administration of metformin and rapamycin can further enhance the immunosuppressive effect of rapamycin. As confirmed in the foregoing examples by the present inventors, metformin has an effect of improving the mitochondrial dysfunction by rapamycin, and thus the co-administration of metformin and rapamycin reduces the side effects of rapamycin and increases the immunosuppressive action of rapamycin, thereby further improving the efficiency of immunosuppressive treatment.

The synergistic effects of immunosuppression or immunoregulation by the co-administration of rapamycin and metformin have been confirmed by the present inventors as follows.

An example of the present invention confirmed that, in the mixed lymphocyte reaction with in vitro allo-response conditions, the simultaneous treatment with rapamycin (1 nM or 100 nM) and metformin (1 mM), compared with the treatment with rapamycin or metformin each, reduced more effectively the proliferation of allogeneic reactive T cells, which are important for the rejection of organ transplantation, and further suppressed the secretion of IFNγ, which is an inflammatory cytokine secreted from allogeneic reactive T cells.

It was confirmed in another example of the present invention that, in T cell activation conditions, the co-treatment with rapamycin (100 nM) and metformin (1 mM), compared with the treatment with rapamycin or metformin each, significantly increased the activity of Treg cells having an inflammation regulating function, and greatly reduced the secretion of IL-17, which is an inflammatory cytokine secreted from pathological cells. It was observed that even the simultaneous treatment with rapamycin and metformin in T cell activation conditions did not induce non-specific cytotoxicity.

In another example of the present invention, the amounts of cytokines and immunoglobulin (IgG) secreted from the splenocytes stimulated with the inflammation-inducing factor LPS were measured. The simultaneous treatment with rapamycin (100 nM) and metformin (1 mM) reduced more effectively the levels of IL-6, TNF-α, and IgG, compared with the treatment with rapamycin or metformin each.

Furthermore, the present inventors confirmed that the co-administration of metformin and rapamycin increased the treatment effect in animal models with arthritis as an autoimmune disease. It was confirmed that, in collagen-induced arthritis mouse models, the rapamycin and metformin co-administered experimental group, compared with rapamycin administered alone group, significantly decreased the occurrence of arthritis and reduced the arthritis index showing the severity of arthritis. It was confirmed that the co-administration of rapamycin and metformin can double the arthritis treatment effect, and can more effectively treat abnormal glucose metabolism caused by arthritis and incidental symptoms, such as obesity and fatty liver, for example, by lowering the blood glucose and the serum lipid content as well as AST and ALT, which are indicators of liver injury.

The above examples show that the simultaneous administration or co-administration of metformin and rapamycin can regulate various immune responses more effectively compared with the administration of metformin or rapamycin alone. Furthermore, it can be seen that metformin has an effect of preventing and/or restoring the rapamycin-induced mitochondrial dysfunction and thus the co-administration of metformin and rapamycin can be effectively used in the immune diseases in need of immunosuppressive or immunoregulatory treatment.

The term “immune disease” refers to a disease induced by dysfunction of the immune system, and may be preferably an immune disease selected from the group consisting of an acute or chronic rejection of organ transplantation, an autoimmune disease, and an inflammatory disease.

The acute or chronic rejection of organ transplantation may be, but is not limited to, an acute or chronic transplantation rejection after the transplantation of heart, lung, heart-lung complex, liver, kidney, pancreas, skin, bowel, or cornea, and a graft-versus-host disease after bone marrow transplantation, especially T cell-mediated rejection after transplantation.

In addition, examples of the autoimmune disease or inflammatory disease may be selected from the group consisting of sepsis, atherosclerosis, bacteremia, systemic inflammatory reaction syndrome, multi-organ dysfunction, osteoporosis, periodontitis, systemic lupus erythematosus, osteoarthritis, rheumatoid arthritis, juvenile chronic arthritis, spondylarthrosis, multiple sclerosis, systemic sclerosis, idiopathic inflammatory muscle disorder, Sjogren's syndrome, systemic angiitis, sarcoidosis, autoimmune hemolytic anemia, autoimmune hemolytic anemia, thyroiditis, diabetes mellitus, immune mediated kidney disease, central and peripheral nervous system demyelinating disorders, idiopathic demyelinating multiple neuritis, Guillain-Barre syndrome, chronic inflammatory demyelinating multiple neuritis, hepatobiliary disease, infectious or autoimmune chronic active hepatitis, primary biliary cirrhosis, granulomatous hepatitis, sclerosing cholangitis, obesity, inflammatory bowel disease (IBD), ulcerative colitis, Crohn's disease, irritable bowel syndrome, gluten-irritable bowel disease, Whipple's disease, autoimmune or immune-mediated skin disease, bullous skin disease, erythema multiforme, contact dermatitis, psoriasis, allergic disease, asthma, allergic rhinitis, atopic dermatitis, food hypersensitivity, acne, urticaria, pulmonary immune disease, eosinophilic pneumonia, idiopathic pulmonary fibrosis, and hypersensitive pneumonia, but are not limited thereto.

In the present invention, metformin and rapamycin or derivatives thereof may be used per se or in the form of a salt thereof, or preferably a pharmaceutically acceptable salt thereof. The term “pharmaceutically acceptable” refers to being physiologically acceptable, and not usually causing an allergic reaction or a similar reaction when administered to humans. An acid added salt formed by a pharmaceutically acceptable free acid is preferable as the salt. An inorganic acid and an organic acid may be used as the free acid. Examples of the organic acid include, but are not limited to, citric acid, acetic acid, lactic acid, tartaric acid, maleic acid, fumaric acid, formic acid, propionic acid, oxalic acid, trifluoroacetic acid, benzoic acid, gluconic acid, methanesulfonic acid, glycolic acid, succinic acid, 4-toluenesulfonic acid, glutamic acid, and aspartic acid. In addition, examples of the inorganic acid include, but are not limited to, hydrochloric acid, bromic acid, sulfuric acid, and phosphoric acid.

In addition, metformin and rapamycin or derivatives thereof may be isolated from nature or may be prepared by chemical synthesis methods known in the art.

The pharmaceutical composition according to the present invention may contain only a pharmaceutically effective amount of mTOR inhibitor and/or metformin or a pharmaceutically acceptable salt thereof or may contain a pharmaceutically acceptable carrier. The term “pharmaceutically effective amount” refers to the amount that shows more effective responses compared with a negative control group, and means the amount sufficient to exhibit the synergistic effect of immunoregulation or immunosuppression through co-administration of an mTOR inhibitor and metformin in the treatment or prevention of an acute or chronic rejection of organ transplantation, an autoimmune disease, or an inflammatory disease, and to allows metformin to alleviate the mTOR inhibitor-induced mitochondrial dysfunction.

A pharmaceutically effective amount of mTOR inhibitor contained as an active ingredient in the pharmaceutical composition is 0.75-16 mg/day/kg of body weight for rapamycin or 5-35 mg/day/kg of body weight for metformin. However, the pharmaceutically effective amount may be properly varied depending on several factors, such as a disease and severity thereof, patient's age, body weight, health conditions, and sex, the route of administration, the treatment period, and the like.

The composition of the present invention may contain an mTOR inhibitor and metformin or a pharmaceutically acceptable salt thereof at a weight ratio of 1:500 to 1:200,000.

The term “pharmaceutically acceptable” composition refers to a non-toxic composition that is physiologically acceptable, does not inhibit an action of an active ingredient when administered to humans, and does not usually induce an allergic reaction or similar reactions, such as gastroenteric troubles and dizziness. The pharmaceutical composition of the present invention may be variously formulated, together with a pharmaceutically acceptable carrier, in order to improve mitochondrial dysfunction or exhibit an immunoregulatory or immunosuppressive effect, depending on the route of administration, by a method known in the art. The carrier includes all kinds of solvents, dispersion media, oil-in-water or water-in-oil emulsions, aqueous compositions, liposomes, microbeads, and microsomes.

The route of administration may be an oral or parenteral route. The parental administration may be, but is not limited to, intravenous, intramuscular, intra-arterial, intramedullary, intradural, intracardiac, transdermal, subcutaneous, intraperitoneal, intranasal, intestinal, topical, sublingual, or rectal administration.

The pharmaceutical composition of the present invention, when orally administered, may be formulated, together with a suitable carrier for oral administration, in the form of a powder, granules, a tablet, a pill, a sugar coated tablet, a capsule, a liquid, a gel, a syrup, a suspension, a wafer, or the like, by a method known in the art. Examples of the suitable carrier may include: sugars including lactose, dextrose, sucrose, sorbitol, mannitol, xylitol, erythritol, and maltitol; starches including corn starch, wheat starch, rice starch, and potato starch; celluloses including cellulose, methyl cellulose, sodium carboxy methyl cellulose, and hydroxypropyl methyl cellulose; and fillers, such as gelatin and polyvinyl pyrrolidone. In some cases, cross-linked polyvinyl pyrrolidone, agar, alginic acid, or sodium alginate may be added as a disintegrant. Furthermore, the pharmaceutical composition of the present invention may further contain an anti-coagulant, a lubricant, a wetting agent, a favoring agent, an emulsifier, and a preservative.

As for the parenteral administration, the pharmaceutical composition of the present invention may be formulated, together with a suitable parenteral carrier, in a dosage form of an injection, a transdermal agent or preparation, and a nasal inhalant, by a method known in the art. The injection needs to be essentially sterilized, and needs to be protected from the contamination of microorganisms, such as bacteria and fungus. Examples of the suitable carrier for the injection may include, but are not limited to, solvents or dispersion media, including water, ethanol, polyols (e. g., glycerol, propylene glycol, liquid polyethylene glycol, etc.), mixtures thereof, and/or vegetable oils. More preferably, Hanks' solution, Ringer's solution, phosphate buffered saline (PBS) or sterile water for injection containing triethanolamine, or an isotonic solution (such as 10% ethanol, 40% propylene glycol, or 5% dextrose) may be used as a suitable carrier. In order to protect the injection from microbial contamination, the injection may further contain various antibiotic and antifungal agents, such as paraben, chlorobutanol, phenol sorbic acid, and thimerosal. In most cases, the injection may further contain an isotonic agent, such as sugar or sodium chloride.

The form of the transdermal agent or preparation includes ointment, cream, lotion, gel, solution for external application, paste, liniment, and aerosol. The term “transdermal administration” means the delivery of an effective amount of an active ingredient, contained in the pharmaceutical composition, into the skin through the topical administration to the skin. For example, the pharmaceutical composition of the present invention is prepared in a dosage form of an injection, which may be then administered by slightly pricking the skin with a 30-gauge needle or being directly applied to the skin. These dosage forms are described in the literature, which is a formulary generally known in pharmaceutical chemistry (Remington's Pharmaceutical Science, 15th Edition, 1975, Mack Publishing Company, Easton, Pa.).

In the case of an inhalation agent or preparation, the compound used according to the invention may be conveniently delivered in the form of an aerosol spray from a pressurized pack or a sprayer, using a suitable propellant, for example, dichlorofluoromethane, trichlorofluoromethane, dichlorotetrafluoroethane, carbon dioxide, or other suitable gas. In the case of a pressurized aerosol, the dosage unit may be determined by providing a valve that delivers a measured quantity. For example, a gelatin capsule and a cartridge used in an inhaler or an insufflator may be formulated to contain a compound, and a powder mixture of proper powder materials, such as lactose or starch.

Other pharmaceutically acceptable carriers may be referenced in the following literature (Remington's Pharmaceutical Sciences, 19th ed., Mack Publishing Company, Easton, Pa., 1995).

In addition, the pharmaceutical composition according to the present invention may further contain one or more buffers (for example, saline solution or PBS), carbohydrates (for example, glucose, mannose, sucrose, or dextran), antioxidants, bacteriostatic agents, chelating agents (for example, EDTA or glutathione), adjuvants (for example, aluminum hydroxide), suspension agents, thickeners, and/or preservatives.

In addition, the pharmaceutical composition of the present invention may be formulated by a method known in the art such that the pharmaceutical composition can provide rapid, sustained, or delayed release of active ingredients after administration into mammals.

In addition, the pharmaceutical composition of the present invention may be administered in combination with a known compound having an effect of improving mitochondrial dysfunction or treating an acute or chronic rejection of organ transplantation, an autoimmune disease, or an inflammatory disease.

In addition, the present invention provides a pharmaceutical complex preparation for treating an immune disease, the pharmaceutical complex preparation being characterized in that: (a) the pharmaceutical complex preparation contains an mTOR inhibitor and metformin or a pharmaceutically acceptable salt thereof at a weight ratio of 1:500 to 1:200,000; and

(b) the mTOR inhibitor and metformin or the pharmaceutically acceptable salt thereof are administered simultaneously, individually, or in a predetermined order.

The mTOR inhibitor may be preferably rapamycin or a derivative thereof.

The pharmaceutical complex preparation of the present invention may be formulated depending on the manner of administration and the route of administration so that the mTOR inhibitor and metformin, which are ingredients, are simultaneously contained in a single dosage form, or the mTOR inhibitor and metformin may be individually formulated and then contained in a single package according to the daily or once-daily dosing unit. The dosage forms of the individually formulated mTOR inhibitor and metformin may or may not be the same as each other. The specific formulation methods of the pharmaceutical complex preparation of the present invention and the specific pharmaceutically acceptable carrier that may be contained in the formulation are as described in the pharmaceutical composition of the present invention described elsewhere herein, and may be referenced in the following literate (Remington's Pharmaceutical Sciences, 19th ed., Mack Publishing Company, Easton, Pa., 1995).

The “pharmaceutically effective amount” refers to the amount that shows more effective reactions compared with a negative control group, and means the amount sufficient to exhibit the synergistic effect of immunoregulation or immunosuppression through co-administration of an mTOR inhibitor and metformin of the pharmaceutical complex preparation of the present invention in the treatment or prevention of an acute or chronic rejection of organ transplantation, an autoimmune disease, or an inflammatory disease, and to alleviate the mitochondrial dysfunction induced by an mTOR inhibitor.

In the case where the mTOR inhibitor of the pharmaceutical complex preparation of the present invention is rapamycin, the daily dose of rapamycin may be 0.75-16 mg/day/kg of body weight, and the dose of metformin or a pharmaceutically acceptable salt thereof may be 5-35 mg/day/kg of body weight.

An mTOR inhibitor and metformin, which are ingredients of the pharmaceutical complex preparation according to the present invention, may be administered simultaneously, individually, or in a predetermined order according to a suitable method. Specific examples of the route of administration are as described above. The term “simultaneous administering” means that the mTOR inhibitor and metformin are taken together or at substantially the same time (for example, the interval of administration time is 15 minutes or less), so that the two ingredients are simultaneously present in the stomach in the case of oral administration. The mTOR inhibitor and metformin, when simultaneously administered, may be formulated such that the mTOR inhibitor and metformin are simultaneously contained in one dosage form. In the case of oral administration, the mTOR inhibitor and metformin may be formulated such that a daily dose of the mTOR inhibitor and metformin are included in a single dose, but are divisionally administered two times, three times, or four times.

A preferable dose of the pharmaceutical complex preparation of the present invention may be appropriately varied according to several factors, such as a disease and the severity thereof, patient's age, body weight, health condition, and sex, the route of administration, and the period of treatment. Since there are individual differences in the bioavailability of the mTOR inhibitor and metformin, it may be preferable to check the concentration of each drug in the blood through an assay based on a monoclonal antibody or the like known in the art, at the initial stage of administering a pharmaceutical preparation of the present invention

The present invention provides a use of metformin or a pharmaceutically acceptable salt thereof for preparing an agent for treating an immunosuppressant-induced mitochondrial disease.

The present invention provides a method for treating an immunosuppressant-induced mitochondrial disease, the method being characterized by administering an effective amount of a composition to a subject in need thereof, the composition comprising, as an active ingredient, metformin or a pharmaceutically acceptable salt thereof.

An embodiment of the present invention is directed to a composition comprising metformin or a pharmaceutically acceptable salt thereof.

Another embodiment of the present invention is directed to a composition consisting of metformin or a pharmaceutically acceptable salt thereof.

Still another embodiment of the present invention is directed to a composition essentially consisting of metformin or a pharmaceutically acceptable salt.

The present invention provides a use of an mTOR inhibitor and metformin or a pharmaceutically acceptable salt thereof for preparing an agent for treating an immune disease.

The present invention provides a method for treating an immune disease, the method being characterized by administering an effective amount of a composition to a subject in need thereof, the composition comprising, as active ingredients, an mTOR inhibitor and metformin or a pharmaceutically acceptable salt thereof.

An embodiment of the present invention is directed to a composition comprising an mTOR inhibitor and metformin or a pharmaceutically acceptable salt thereof.

Another embodiment of the present invention is directed to a composition consisting of an mTOR inhibitor and metformin or a pharmaceutically acceptable salt thereof.

Still another embodiment of the present invention is directed to a composition essentially consisting of an mTOR inhibitor and metformin or a pharmaceutically acceptable salt.

The term “effective amount” refers to the amount showing an effect of alleviating, treating, preventing, detecting or diagnosing an immunosuppressant-induced mitochondrial disease or an immune disease, and the term “subject” refers to an animal, preferably, a mammal, and especially, an animal including a human being, and may be a cell, tissue, and organ, or the like, originating from an animal. The subject may be a patient in need of treatment.

The term “treatment” or “treat” refers to inhibiting occurrence or recurrence of diseases, alleviating symptoms, reducing direct or indirect pathological consequences of diseases, reducing the disease progression rate, alleviating, improving, or relieving disease conditions, or showing improved prognosis. More specifically, the term “treatment” of the present invention refers broadly to alleviating the symptoms of an immunosuppressant-induced mitochondrial disease or an immune disease, and may include curing or substantially preventing such a disease or improving the condition thereof, and includes, but not limited to, relieving, curing, or preventing one symptom or most of the symptoms resulting from an immunosuppressant-induced mitochondrial disease or an immune disease.

The term “comprising” is used synonymously with “containing” or “being characterized”, and does not exclude additional ingredients or steps that are not mentioned in the compositions and the methods. The term “consisting of” excludes additional elements, steps, or ingredients that are not separately described. The term “essentially consisting of” means that in the scope of the compositions or methods, the term includes described materials or steps as well as any material or step that does not substantially affect basic characteristics of the compositions or methods.

### Advantageous Effects

Therefore, the present invention provides a composition comprising metformin for improving mitochondrial functions impaired by an immunosuppressant, a pharmaceutical composition comprising, as active ingredients, metformin and a mammalian target of rapamycin inhibitor (mTOR inhibitor) for preventing or treating an immune disease, and a pharmaceutical complex preparation. The composition of the present invention effectively mitigates the mitochondrial function impairment caused by side effects of an existing immunosuppressant and further improves an immunosuppressive treatment effect, and thus can be favorably used in the prevention or treatment of a rejection of transplantation, an autoimmune disease, an inflammatory disease, and the like, in need of immunosuppression.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A AND 1B show mitochondrial oxygen consumption rate (OCR) measurement experiments illustrating the effect of rapamycin on mitochondrial respiration. The horizontal axis represents time (min) and the vertical axis represents OCR (pmol/min). In FIGS. 1A and 1B, “Control” indicates experiment results for a negative control group, “Control+Rapamycin” indicates experiment results for cells treated with rapamycin alone, and “Control+Rapamycin+Metformin” indicates experiment results for cells co-treated with rapamycin and metformin.

FIG. 2 shows fluorescence microscopic images of mitochondria stained with MitoTracker, illustrating the effect of metformin and rapamycin on mitochondrial content. Red indicates mitochondria (MitoTracker), green indicates α-tubulin, and blue indicates DAPI. Nil indicates a control group. The mitochondrial content determined as the mean fluorescence intensity (MFI) was quantified by the graph below.

FIG. 3 shows fluorescent microscopic images of JC-1 staining, illustrating the effect of metformin and rapamycin on the mitochondrial membrane potential. The mitochondrial membrane potential determined as the mean fluorescence intensity (MFI) was quantified by the graph below.

FIG. 4 shows the real-time RT-PCR results illustrating the effect of metformin and rapamycin on the expression of Ndufb5, Uqcrb, and Cycs, which are genes associated with mitochondrial functions.

FIG. 5 shows a schedule of an animal experiment using rats to investigate the effect of co-administration of metformin on the rapamycin-induced diabetic side effects.

FIGS. 6A AND 6B illustrate the body weight (FIG. 6A) and 24 hour-urine volume (FIG. 6B) of rats in a drug-untreated control group (VH), a rapamycin administered group (Rapa), and a rapamycin and metformin co-administered group (Rapa+Met) according to experimental conditions shown in the schedule in FIG. 5.

FIGS. 7A AND 7B show a graph represented by the change in the blood glucose level over time (min) (FIG. 7A) and a bar graph using the area (AUCg) of the above graph (FIG. 7B) for the intraperitoneal glucose tolerance test results of rats in a drug-untreated control group (VH), a rapamycin administered group (Rapa), and a rapamycin and metformin co-administered group (Rapa+Met) according to experimental conditions shown in the schedule in FIG. 5.

FIGS. 8A AND 8B show a graph represented by the change in the blood glucose level over time (min) (FIG. 8A) and a bar graph using the area (AUCg) of the above graph (FIG. 8B) for the insulin resistance test results of rats in a drug-untreated control group (VH), a rapamycin administered group (Rapa), and a rapamycin and metformin co-administered group (Rapa+Met) according to experimental conditions shown in the schedule in FIG. 5.

FIG. 9 shows test results illustrating the effects of metformin and rapamycin on allogeneic reactive T cells in the mixed lymphocyte reaction. *p<0.05

FIG. 10 shows ELISA test results illustrating the effects of metformin and rapamycin on the amount of inflammatory cytokine IFN-γ secreted in allogeneic reactive T cells in the mixed lymphocyte reaction.

FIG. 11 shows MTT test results for measuring cytotoxicity of metformin and rapamycin in the splenocytes in T-cell activation conditions.

FIG. 12 shows ELISA test results illustrating the effect of metformin and rapamycin on the expression level of inflammatory cytokine IL-17 in splenocytes in T-cell activation conditions. *p<0.05

FIGS. 13A AND 13B show flow cytometry results illustrating the effects of metformin and rapamycin on the activity of Treg cells in splenocytes in T-cell activation conditions. FIG. 13A shows flow cytometry data obtained by gating and analyzing cells expressing CD25 and Foxp3, and FIG. 13B shows a bar graph illustrating the proportion of Foxp3+CD25+.

FIGS. 14A AND 14B show ELISA test results illustrating the effect of metformin and rapamycin on the amounts of inflammatory cytokines IL-6 (FIG. 14A) and TNF-α (FIG. 14B) secreted in splenocytes stimulated with LPS. *p<0.05

FIG. 15 shows ELISA test results illustrating the effects of metformin and rapamycin on the amounts of immunoglobulin (IgG) secreted in splenocytes stimulated with LPS. *p<0.05

FIGS. 16A AND 16B show the arthritis score (FIG. 16A) and the incidence (%, FIG. 16B) over time in a drug-untreated control group (Vehicle), a rapamycin administered group (Rapamycin), and a rapamycin and metformin co-administered group (Met+Rapa) in collagen-induced arthritis mouse models.

FIGS. 17A AND 17B show blood glucose test results (FIG. 17A) and insulin resistance test results (FIG. 17B) in a drug-untreated control group (Vehicle), a rapamycin administered group (Rapa), and a rapamycin and metformin co-administered group (M+R) in collagen-induced arthritis mouse models.

FIGS. 18A AND 18B show blood glucose and blood lipid test results (FIG. 18A) and liver injury indexes AST and ALT level measurement results (FIG. 18B) for confirming fatty liver improvement effects in a drug-untreated control group (Vehicle), a rapamycin administered group (Rapa), and a rapamycin and metformin co-administered group (M+R) in collagen-induced arthritis mouse models.

### MODE FOR CARRYING OUT THE INVENTION

Hereinafter, the present invention will be described in detail.

The following examples are merely for illustrating the present invention and are not intended to limit the scope of the present invention.

### EXAMPLE 1

Effect of Metformin on Rapamycin-Induced Mitochondrial Dysfunction

<1-1> Measurement of Mitochondrial Respiration

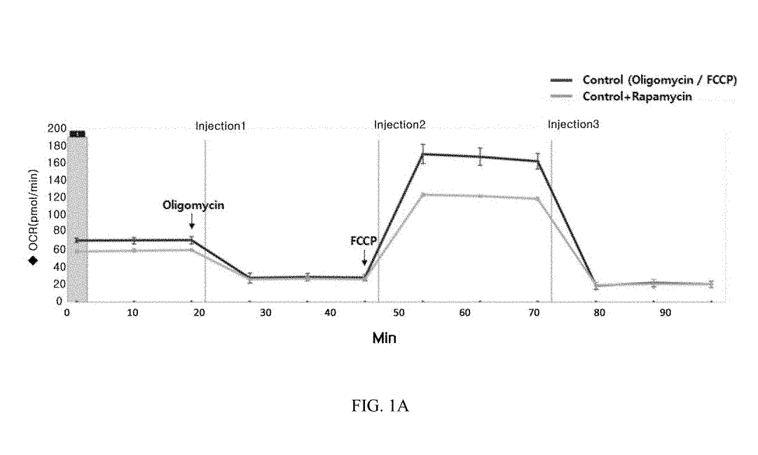

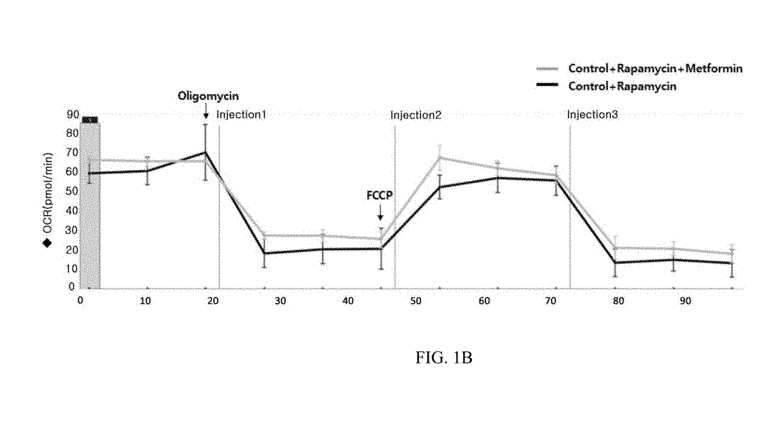

The mitochondrial respiration was measured to investigate the effect of rapamycin on mitochondrial function FIGS. 1A AND 1B.

Synovial cells isolated from patients with rheumatoid arthritis (RA) were treated with rapamycin (100 nM) according to test conditions, and then the cells were treated with oligomycin (2 uM) to reduce the respiratory function at the initial stage of mitochondrial respiration measurement, and were treated with FCCP (3 uM) to increase mitochondrial respiration at the middle stage of the measurement, so that the change in mitochondrial respiratory rate was observed through the measurement of the oxygen consumption rate (OCR).

As shown in FIG. 1A, the experiment group treated with rapamycin showed reduced respiration capacity compared with the control group before the treatment with oligomycin, and the increase in mitochondrial respiratory rate by FCCP was also confirmed to be remarkably low in the experiment group treated with rapamycin compared with the control group. Therefore, it can be seen that the immunosuppressive function of rapamycin can alleviate inflammation responses, but rapamycin may cause the dysfunction of reducing the mitochondrial respiration.

The present inventors examined the effect of metformin on rapamycin-induced mitochondrial respiration deterioration. The cells were treated with rapamycin (100 nM) alone or co-treated with rapamycin (100 nM) and metformin (1 mM), and then the mitochondrial respiratory rate was examined through the measurement of the oxygen consumption rate. Oligomycin and FCCP treatment conditions were the same.

As shown in FIG. 1B, the experiment group co-treated with rapamycin and metformin was observed to have an increased mitochondrial respiratory rate compared with the treatment with rapamycin alone before and after the oligomycin treatment. In addition, the increase in mitochondrial respiratory rate by FCCP was also observed to be higher in the co-treatment with rapamycin and metformin. That is, metformin has an effect of mitigating the rapamycin-induced reduction in mitochondrial respiration in the co-treatment with rapamycin. Therefore, it was confirmed that metformin can increase the inflammation inhibitory effect by co-administration with rapamycin and can improve the rapamycin-induced mitochondrial dysfunction.

<1-2> Analysis of Mitochondrial Content

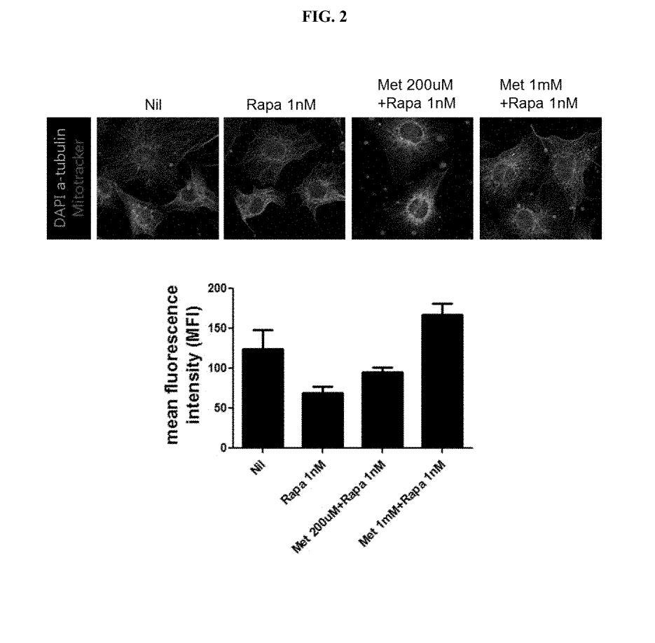

The present inventors examined the effect of metformin to improve the rapamycin-induced mitochondrial dysfunction by observing the mitochondrial content (FIG. 2).

NIH3T3 cells were treated with metformin (200 uM or 1 mM) and rapamycin (1 nM), and after 72-hr incubation, mitochondria were stained with MitoTracker, and α-tubulin staining was allowed to show the overall morphology of the cells. The mitochondria were observed by fluorescent microscopy for each experimental condition. Specifically, the MitoTracker was diluted in DMEM medium to a concentration of 100 nM, and then added on the NIH3T3 plate, followed by incubation at 37° C. for 15 minutes and washing with PBS. Thereafter, for α-tubulin staining, the cells were fixed with acetone and methanol (1:1) for 15 minutes, and then washed with PBS for 15 minutes. The cells were blocked with 10% normal goat serum for 30 minutes, and then incubated with α-tubulin (1:500) antibody at 4⊏ overnight, washed with PBS, stained with DAPI (1:500), and then observed by fluorescent microscopy.

As shown in FIG. 2, the mitochondrial content was reduced due to side effects of mitochondrial respiration suppression in the cells treated with rapamycin compared with a drug-untreated negative control group (Nil). On the contrary, it was confirmed that the mitochondrial content was greatly increased in the cells co-treated with rapamycin and metformin compared with the cells treated with rapamycin. That is, it was confirmed that the co-treatment with metformin and rapamycin had an effect of mitigating the rapamycin-induced reduction in mitochondrial content.

<1-3> Analysis of Mitochondrial Membrane Potential

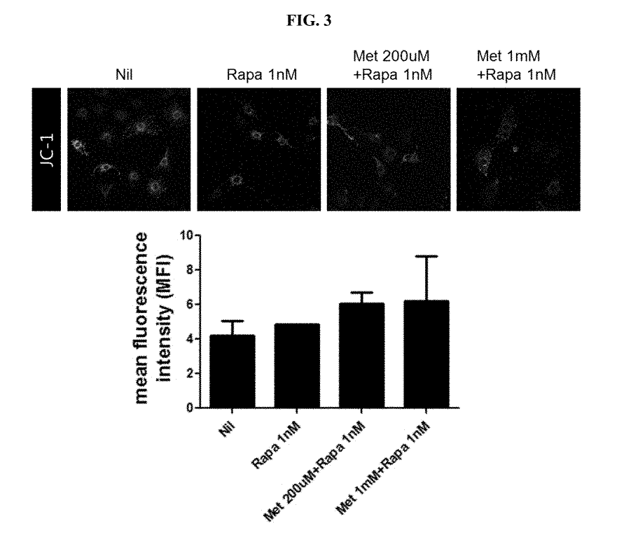

The present inventors examined the effect to metformin to improve the rapamycin-induced mitochondrial dysfunction by observing the mitochondrial membrane potential (FIG. 3).

NIH3T3 cells were treated with metformin (200 uM or 1 mM) and/or rapamycin (1 nM) according to experimental conditions, and after 72-hr incubation, JC-1 staining for showing the mitochondrial membrane potential was conducted, and the change in mitochondrial membrane potential was observed by fluorescent microscopy for each experimental condition. For JC-1 staining, the cells were incubated in JC-1, which had been diluted in DMEM to a final concentration of 100 nM, at 37□ for 15 minutes, and then the exchange into new DMEM medium was conducted, followed by observation by fluorescent microscopy.

As shown in FIG. 3, it was confirmed that the mitochondria of the cells untreated with any drug (Nil) were stained with red fluorescence since the mitochondrial membrane potential was well maintained, but the green fluorescence was increased in the rapamycin treatment condition and thus the mitochondrial membrane potential was not normally maintained. Meanwhile, it was observed that in the cells co-treated with metformin and rapamycin, the membrane potential was restored and thus red fluorescence was greatly increased. That is, it was confirmed that the co-treatment with metformin and rapamycin can alleviate the rapamycin-induced mitochondrial membrane potential damage.

<1-4> Expression of Mitochondria Function-Related Genes

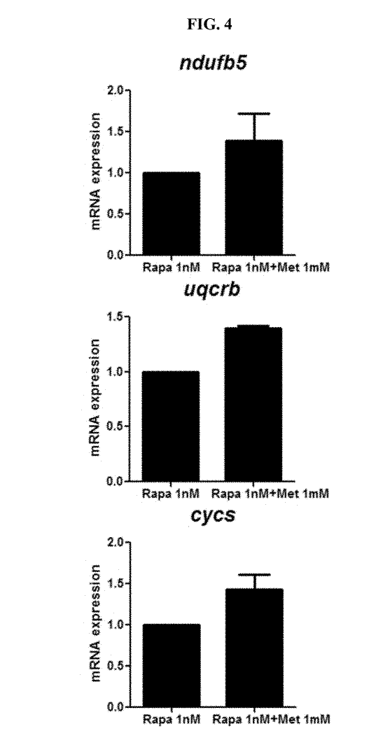

The present inventors examined the effect of metformin to improve the rapamycin-induced mitochondrial dysfunction through the expression patterns of genes, which are important for mitochondrial functions (FIG. 4).

NIH3T3 cells were incubated according to each experimental condition (rapamycin 1 nM, metformin 200 uM or 1 mM) for 3 days, and then total RNA was extracted from the cells, and the expression patterns of the Ndufb5, Uqcrb, and Cycs genes associated with the mitochondrial potential maintenance or mitochondrial respiration functions were observed by real-time PCR (RT-PCR). Primer nucleotide sequences used for RT-PCR are as described in table 1.

TABLE 1
Primer nucleotide sequences

| Gene/ | | |
|---|---|---|
| Direction | forward | reverse |
| Ndufb5 | TCCCAGAAGGCTACATCCCT | ATTCCGGGCGATCCATCTTG |
| | (SEQ ID NO: 1) | (SEQ ID NO: 2) |
| Uqcrb | TCAAGCAAGTGGCTGGATGG | TCAGGTCCAGGGCTCTCTTA |
| | (SEQ ID NO: 3) | (SEQ ID NO: 4) |
| Cycs | AATCTCCACGGTCTGTTCGG | GGTCTGCCCTTTCTCCCTTC |
| | (SEQ ID NO: 5) | (SEQ ID NO: 6) |

It was confirmed in FIG. 4 that the expression levels of Ndufb5, Uqcrb, and Cycs genes were increased in the co-treatment with metformin and rapamycin compared with the treatment with rapamycin alone. That is, metformin improves the expression of genes associated with mitochondrial functions, and thus presents the possibility of exhibiting the effect of improving the rapamycin-induced mitochondrial dysfunction.

### EXAMPLE 2

Rapamycin-Induced Diabetic Symptoms and Effect of Metformin Co-Administration

In order to examine the body side effects related to rapamycin-induced mitochondrial dysfunction, the present inventors observed the changes in the body metabolism after the administration of rapamycin into rats, and examined the effects of the co-administration of rapamycin and metformin.

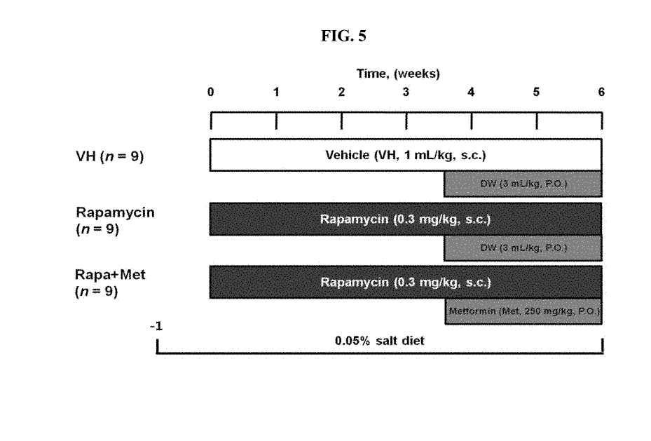

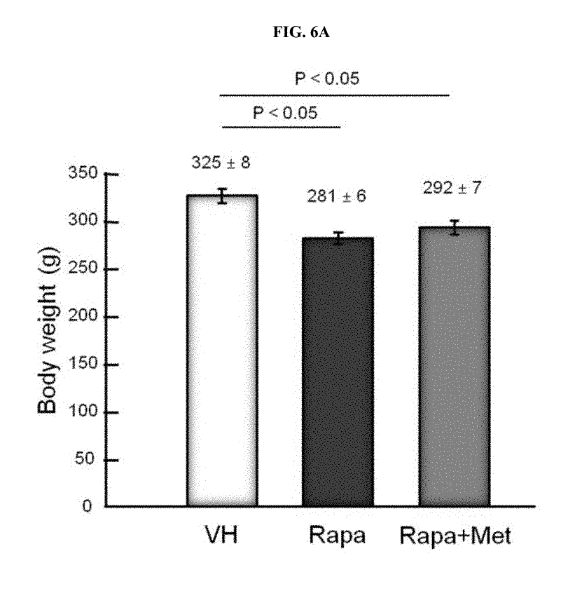

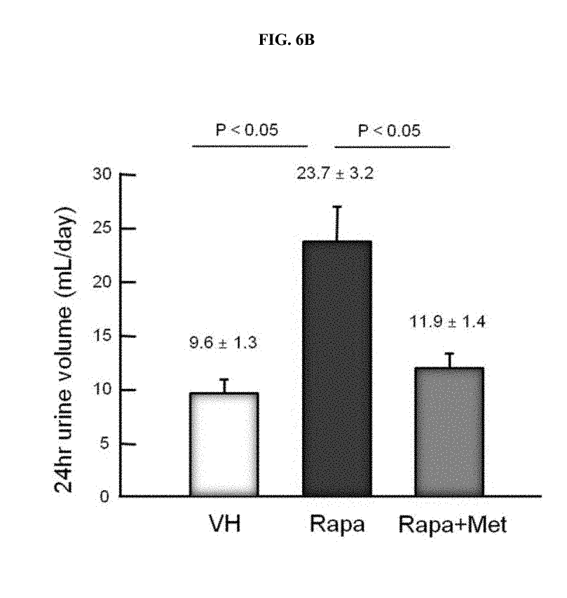

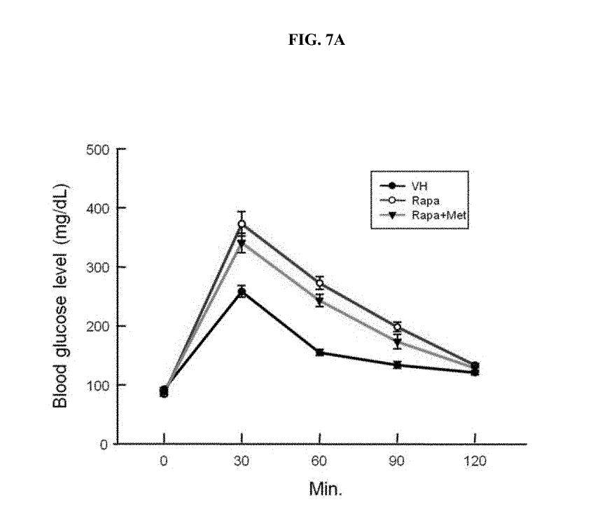

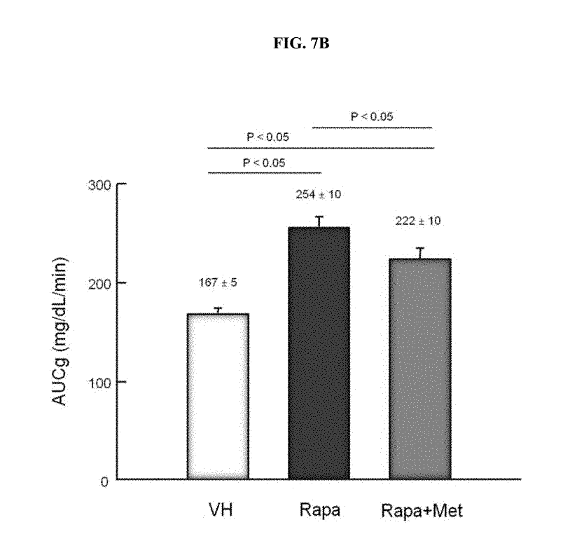

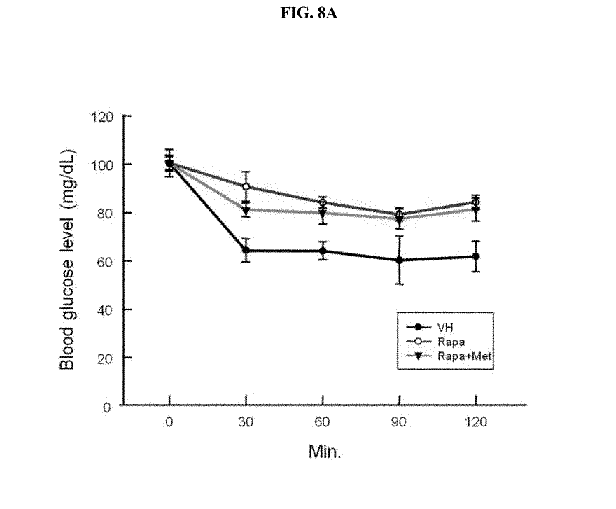

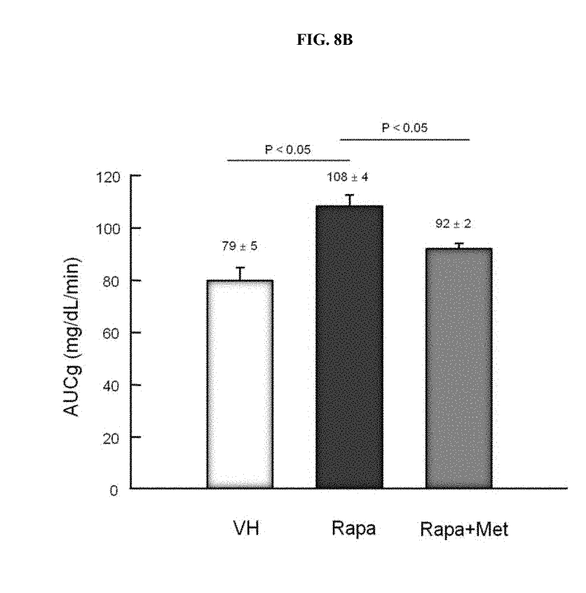

As experimental animals, Sprague-Dawley rats with 200-220 grams were used. The animals were fed with a 0.05% low-salt diet, and the experiment was conducted for a total of 6 weeks while drugs were administered according to experimental conditions (FIG. 5). The animals were divided into three groups—a vehicle group (VH) as a control group, and a rapamycin administered alone group (Rapamycin) and a rapamycin and metformin co-administered group (Rapa+Met) as experimental groups, and each group included nine rats. Rapamycin dissolved in olive oil was subcutaneously injected into the rapamycin administered alone group (Rapamycin) and the co-administered group (Rapa+Met) at a dose of 0.3 mg/kg of body weight every day for six weeks. Metformin was orally administered at a dose of 250 mg/kg of body weight every day for 2.5 weeks from the 3.5 weeks of rapamycin administration. Distilled water (DW, 3 mL/kg) instead of metformin was orally administered into the rapamycin administered alone group and the control group. The body weight and 24-hr urine volume of the control and experimental groups were measured six weeks after the start of the experiment FIGS. 6A and 6B. The urine volume was measured in the metabolic cage. The animals of each group were tested for the intraperitoneal glucose tolerance test (IPGTT) and the insulin tolerance test (ITT), and the change in blood glucose level over time was observed FIGS. 7A, 7B, 8A and 8B). The intraperitoneal glucose tolerance test (IPGTT) was performed by intraperitoneal administration of 1.5 g/kg of body weight of glucose after fasting. The insulin resistance test was carried out such that the blood glucose level was measured at 30-min intervals after subcutaneous injection of insulin at 0.8 U/kg of body weight following 5-hr fasting. The quantitative index (area under the curve of glucose, AUCg) was derived using a graph of blood glucose level change per hour, and was expressed as a bar graph. Data were expressed as mean value±standard deviation, and statistical significance was determined by student's t-test.

<2-1> Changes in Body Weight and Urine Volume

As shown in FIG. 6A, as a result of measuring the body weight 2.5 weeks after the metformin administration, the rapamycin administered alone group (Rapa) and the co-administered group (Rapa+Met) showed significant reductions compared with the control group (VH). As shown in FIG. 6B, the 24-hr urine volume was significantly increased in the rapamycin administered alone group compared with the control group, but was maintained at similar levels in the co-administered group and the control group. It was confirmed that the co-administration of rapamycin and metformin can prevent the rapamycin-induced urine volume change.

The body weight reduction and the urine volume increase, induced by the rapamycin administration, correspond to representative initial diabetic symptoms shown with the increase of blood glucose. Therefore, the glucose metabolic activity of the mice of the control group and the experiment groups was examined by the glucose tolerance test and insulin resistance test.

<2-2> Glucose Tolerance Test

As shown in FIG. 7A, as a result of the intraperitoneal glucose tolerance test (IPGTT), the rapamycin administered alone group (Rapa) maintained the highest blood glucose level among the three groups. It can be seen that the blood glucose level of the metformin co-administered group (Rapa+Met) was higher than that of the control group (VH), but was lower than that of the rapamycin administered alone group. It could be confirmed that, also in the glucose per unit volume, derived from the area under the curve of glucose (AUCg) in the IPGTT result graph shown in FIG. 7B, the blood glucose level was statistically significantly reduced in the metformin co-administered group compared with the rapamycin administered alone group.

<2-3> Insulin Tolerance Test

As shown in FIG. 8A, as a result of the insulin tolerance test (ITT), the rapamycin administered alone group (Rapa) maintained the highest blood glucose level among the three groups. It could be confirmed that the blood glucose level of the metformin co-administered group (Rapa+Met) was also higher than that of the control group (VH), but was lower than that of the rapamycin administered alone group. It could be confirmed that, also in the glucose per unit volume, derived from the area under the curve of glucose (AUCg) in the ITT result graph shown in FIG. 8B, the blood glucose level was statistically significantly reduced in the metformin co-administered group compared with the rapamycin administered alone group.

It was confirmed through the above animal experimental results that the administration of rapamycin induces diabetic symptoms and the co-administration of metformin with rapamycin can alleviate diabetic symptoms.

### EXAMPLE 3

Effect of Metformin and Rapamycin on Allogeneic Immune Response

<3-1> Analysis of Allogeneic Reactive T Cell Proliferation

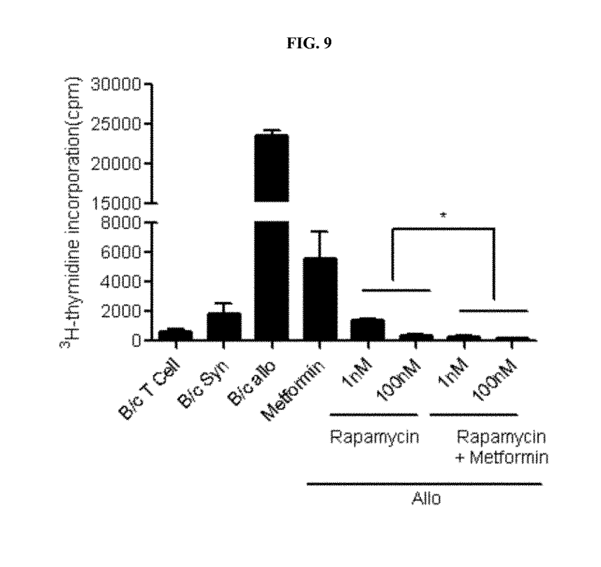

The increase effect of immunoregulatory capacity by simultaneous treatment with metformin and rapamycin was examined in the in vitro allo-response conditions. The effect of metformin and rapamycin on allogeneic reactive T cell proliferation was examined through the mixed lymphocyte reaction (MLR) (FIG. 9).

CD4+ T cells (2×10⁵ cells/well) from normal donors (Balb/c, responder) and splenocytes (2×10⁵ cells/well) with T cells removed, which were derived from irradiated recipients (syngeneic) or donors (C57BL/ 6, stimulator, allogeneic), were added to each well of a 96-well plate, followed by mixed incubation. Here, for the allogeneic reaction, the cells were treated with metformin (1000 uM) and rapamycin (1 nM or 100 nM) according to experimental conditions, followed by incubation for 3 days. On the last day of the incubation, [³H]-thymidine was added, followed by additional incubation for 18 hours, and the [³H]-thymidine uptake of cells was measured by the Liquid Scintillation Counter (Beckman, USA), and expressed as cpm. Statistical assay was conducted by using Graph prism (t-test, ANOVA), and values of p<0.05 were considered statistically significant.

As shown in FIG. 9, the results confirmed that the treatment with metformin alone or the treatment with rapamycin alone reduced the cpm values due to the absorption of [³H]-thymidine, thereby inhibiting T cell proliferation. Such a T cell proliferation inhibitory effect was more significant in the simultaneous treatment with metformin and rapamycin. That is, it can be seen that the co-administration of metformin and rapamycin maximizes the effect of inhibiting the allogeneic reactive T cell proliferation.

<3-2> Measurement of Amount of Inflammatory Cytokine Secreted in Allogeneic Reactive T Cells

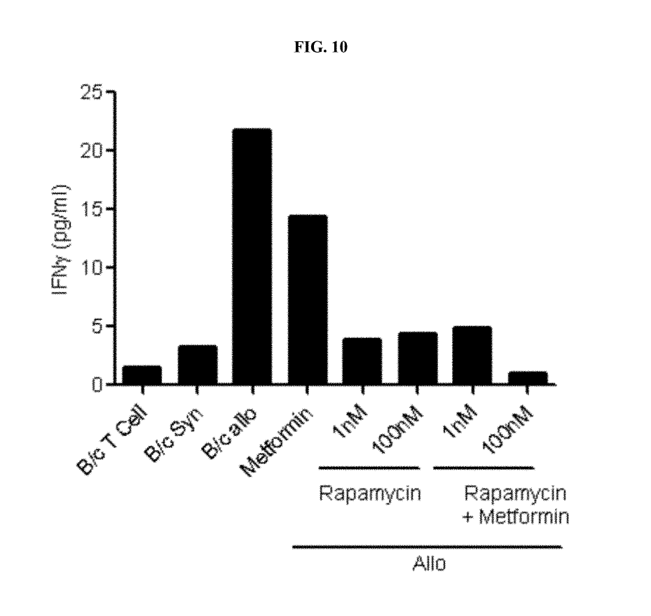

The effect of metformin and rapamycin on the secretion of inflammatory cytokines by allogeneic reactive T cells (FIG. 10).

In the same in vitro allo-response conditions as in example <3-1>, the cells were treated with metformin (1000 μM) or rapamycin (1 nM or 100 nM) according to experimental conditions, followed by incubation for 3 days, and then the amount of IFN-γ secreted in the culture liquid obtained by the incubation for 3 days was measured by ELISA.

As shown in FIG. 10, it was observed that the secretion of IFN-γ was reduced by the treatment with metformin and rapamycin alone, but such an IFN-γ secretion inhibitory effect was more remarkable in the co-treatment with metformin and rapamycin (Rapamycin+Metformin 100 nM). That is, it can be seen that, in the mixed lymphocyte reaction conditions, the co-treatment with metformin and rapamycin can inhibit more effectively the secretion of inflammatory cytokine of allogeneic reactive T cells.

### EXAMPLE 4

Effect of Metformin and Rapamycin on T Cell Activity

<4-1> Assessment of Non-Specific Cytotoxicity in T Cell Activation Conditions

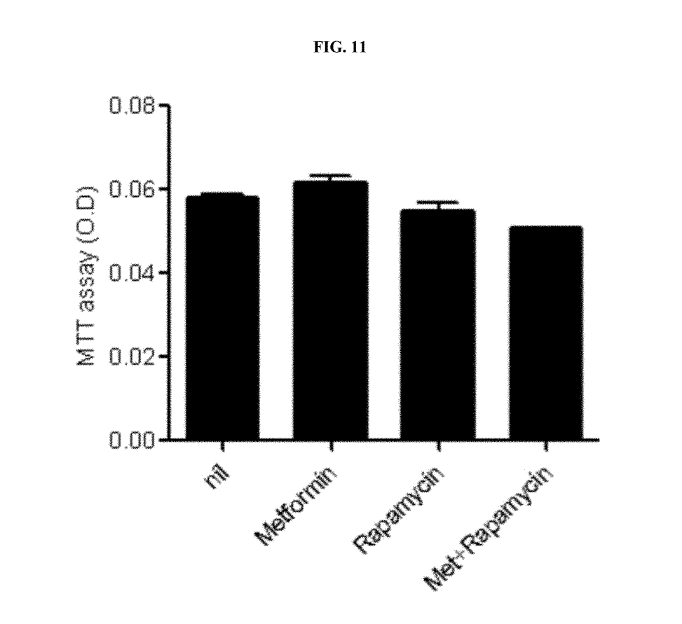

It was examined through the MTT experiment whether metformin and rapamycin show non-specific cytotoxicity on activated T cells (FIG. 11).

Splenocytes obtained from normal C57BL/6 mice were added at 2×10⁵ cells in the 96-well plate, and treated with metformin (1000 μM) or rapamycin (100 nM) according to experimental conditions, followed by incubation for 3 days, under anti-CD3 activation conditions (0.5 μg/ml). For MTT analysis, 3-(4,5-dimethyl-thiazol-2-yl)-2,5-diphenyltetrazolium bromide was added 4 hours before cell harvest, followed by incubation for 4 hours, and then each well was treated with DMSO, and the absorbance was measured at the wavelength of 540 nm.

As shown in FIG. 11, neither the treatment with metformin (Metformin) or rapamycin (Rapamycin) alone nor the co-treatment with metformin and rapamycin (Met+Rapamycin) showed a great difference from the control group. Therefore, it was confirmed that there was no non-specific cytotoxicity of drug treatment according to experimental conditions.

<4-2> Measurement of Secretion Amount of Inflammatory Cytokine IL-17

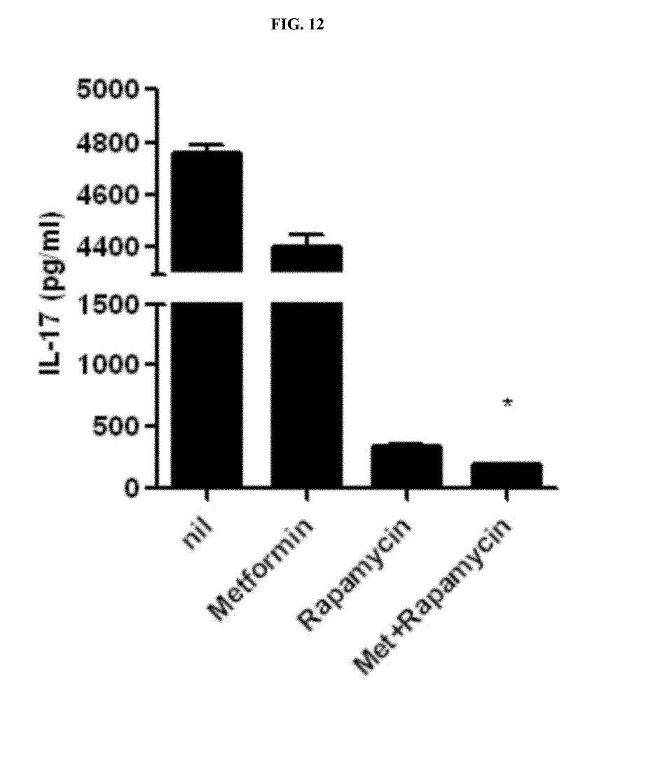

The effect of metformin and rapamycin on the expression of the inflammatory cytokine IL-17 was examined (FIG. 12).

Rat splenocytes were obtained in the same manner as in example <4-1>, and incubated in T cell activation conditions (anti-CD3 0.5 μg/ml). The cells were treated with metformin (1000 uM) or rapamycin (100 nM) according to experimental conditions, followed by incubation for 3 days, and then the amount of IL-17 present in the culture liquid was measured by ELISA.

As shown in FIG. 12, the amount of IL-17 present in the culture liquid was reduced in the treatment with metformin (Metformin) or rapamycin (Rapamycin) alone compared with the treatment without any drug (Nil), but was more remarkably reduced in the co-treatment with metformin and rapamycin (Met+Rapamycin). That is, it can be seen that the co-treatment with metformin and rapamycin can inhibit more effectively the expression of the inflammatory cytokine secreted in T cells.

<4-3> Analysis of Treg Cell Activity

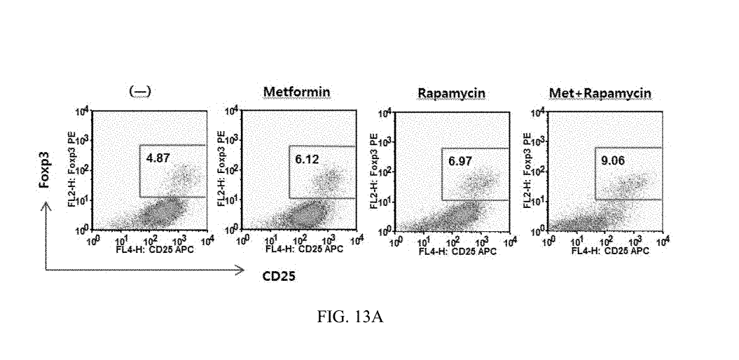

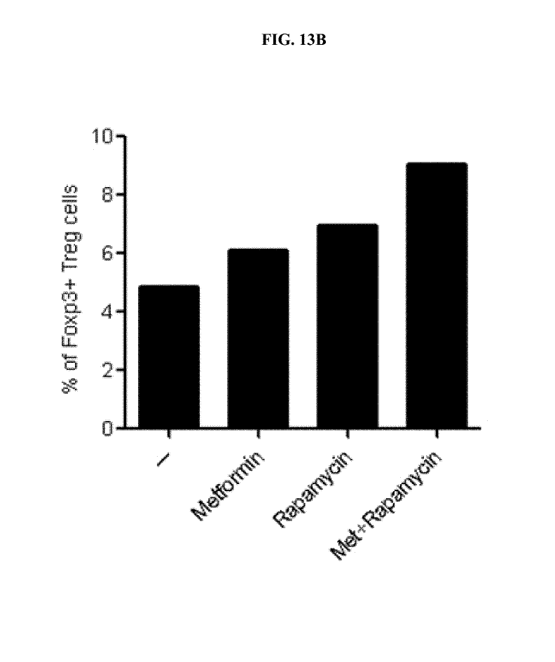

The effect of metformin and rapamycin on Treg cell activity was examined (FIGS. 13A and 13B).

Splenocytes obtained from normal C57BL/6 mice were added at 1×10⁶ cells in the 24-well plate, and treated with metformin (1000 μM) or rapamycin (100 nM) according to experimental conditions, followed by incubation for 3 days, under anti-CD3 activation conditions (0.5 μg/ml). For flow cytometry, the cells were treated with anti-CD4-percp antibody and anti-CD25-APC antibody, followed by incubation at 4□ for 30 minutes, and then the cells were subjected to permeabilization, and then reacted with anti-Foxp3-PE. For analysis of Treg activity, the cells expressing the CD4+CD25+Foxp3+ marker were gated and analyzed. The results were expressed by a bar graph showing the proportion of Foxp3+CD25+ cells occupying in the cultured all CD4+ cells.

As shown in FIGS. 13A and 13B, the Treg activity was increased in the treatment with metformin (Metformin) or rapamycin (Rapamycin) alone compared with the control group (-), but the Treg activity was more remarkably increased in the co-treatment with metformin and rapamycin (Met+Rapamycin). That is, it can be seen that the co-treatment with metformin and rapamycin further increased the Treg activating effect of each drug.

### EXAMPLE 5

Effect of Metformin and Rapamycin on Inflammation Response

<5-1> Measurement of Inflammatory Cytokines

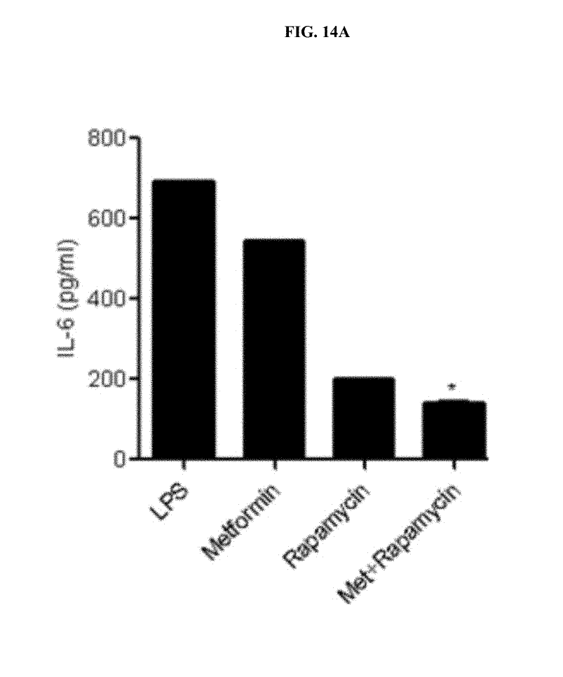

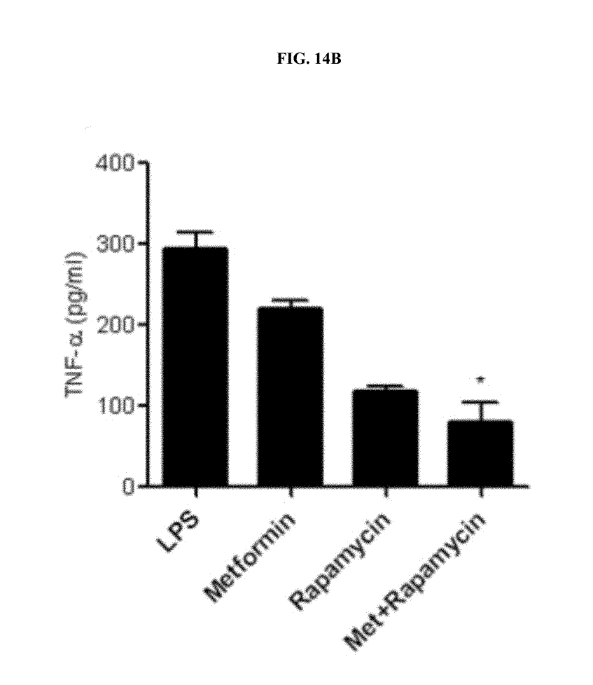

In order to examine the effect of metformin and rapamycin on the activity of inflammatory cytokines, the amounts of inflammatory cytokines secreted in LPS-stimulated splenocytes were examined (FIGS. 14A and 14B).

Splenocytes obtained from normal C57BL/6 mice were added into a 24-well plate (1×10⁶ cells/well), and stimulated by LPS (100 ng/ml), and then treated with metformin (1000 uM) or rapamycin (100 nM) according to experimental conditions, followed by incubation for 3 days. The concentrations of IL-1β and TNF-α present in the culture liquid were measured by ELISA. Statistical analysis was conducted by using Graph prism (t-test, ANOVA), and values of p<0.05 were considered statistically significant.

As shown in FIGS. 14A and 14B, the concentrations of IL-6 (FIG. 14A) and TNF-α (FIG. 14B) present in the culture liquid were reduced in the treatment with metformin (Metformin) or rapamycin (Rapamycin) alone compared with the control group (LPS). The concentrations of IL-6 and TNF-α were further reduced in the co-treatment with metformin and rapamycin (Met+Rapamycin) compared with metformin and rapamycin alone. That is, it can be seen that the co-treatment with metformin and rapamycin inhibited more effectively the secretion of inflammatory cytokines in the inflammation induced situations.

<5-2> Measurement of Immunoglobulin

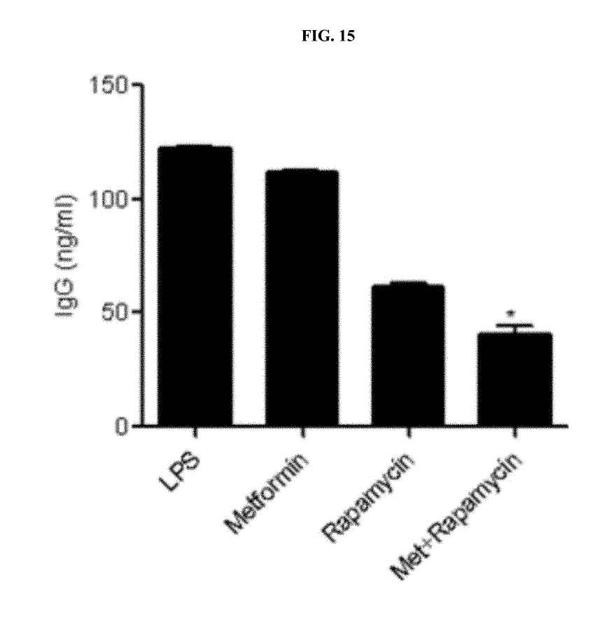

In order to examine the effect of metformin and rapamycin to regulate the inflammation response, the amount of immunoglobulin (IgG) in the culture liquid of the LPS-stimulated splenocytes was measured (FIG. 15).

Mouse splenocytes were incubated by the same method as in example <5-1>, and stimulated by LPS, and treated with metformin or rapamycin at the same concentration as in example <5-1> according to experimental conditions. Thereafter, the level of IgG present in the culture liquid was measured by ELISA.

As shown in FIG. 15, the concentration of immunoglobulin present in the culture liquid was reduced in the treatment with metformin (Metformin) or rapamycin (Rapamycin) alone compared with the control group (LPS), but the amount of immunoglobulin was more significantly reduced in the co-treatment with metformin and rapamycin. It can be seen that the co-treatment with metformin and rapamycin can more effectively regulate inflammation, as evidenced by the reduction in the amount of immunoglobulin.

### EXAMPLE 6

Effect of Rapamycin and Metformin Co-Administration on Arthritis

<6-1> Arthritis Index and Incidence of Arthritis Animal Models

The treatment effect of the rapamycin and metformin co-administration in the autoimmune disease animal models was examined. To this end, the effects between rapamycin alone administration and rapamycin and metformin co-administration in the obesity arthritis mouse models induced by a high-fat diet simultaneously with collagen were compared.

The arthritis-induced mouse models were fabricated by the subcutaneous injection of chicken type II collagen (100 μg/mouse) together with the supply of a high-fat diet (60 kcal). One week after the collagen injection, the mice were orally administered with a single or complex preparation containing rapamycin (1 mg/kg) alone or containing rapamycin (1 mg/kg) and metformin (50 mg/kg) together, and then the arthritis index and incidence were observed for 12 weeks.

The arthritis score was calculated as the mean between observers for the values obtained by adding up the scores per animal, obtained according to the scale below. Scores and criteria for arthritis evaluation are as follows: zero point for no edema or swelling; 1 point for mild edema and rubefaction restricted to feet or ankle joints; 2 points for slight edema and rubefaction from ankle joints to metatarsal; 3 points for moderate edema and rubefaction from ankle joints to metatarsal; 4 points for edema and rubefaction from ankles to the whole leg. The incidence was calculated by estimating four swollen feet of a mouse to 100% and one swollen foot to a mouse to 25%.

As can be confirmed in FIGS. 16A and 16B, the arthritis score (FIG. 16A) and the arthritis incidence (FIG. 16B) were lower in the co-administration with metformin and rapamycin compared with the treatment with rapamycin alone.

<6-2> Glucose Tolerance Test (GTT) and Insulin Tolerance Test (ITT) in Arthritis Animal Models

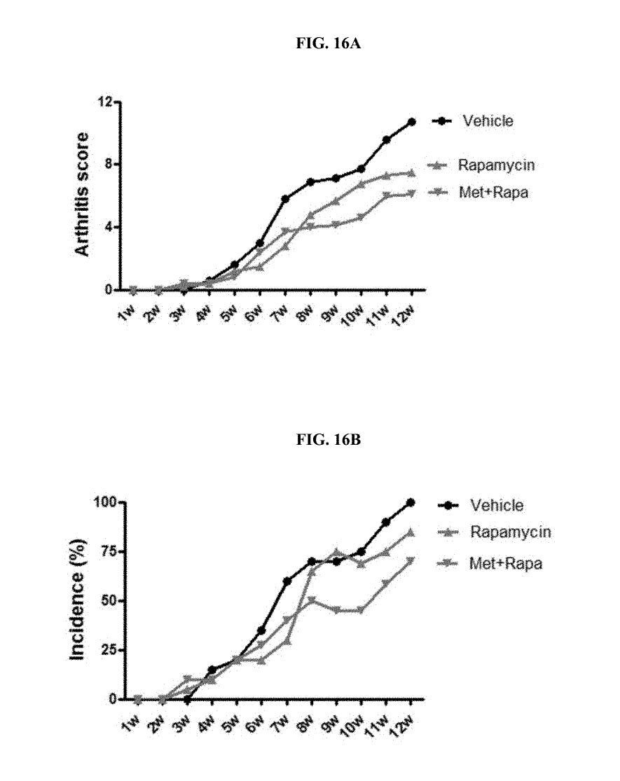

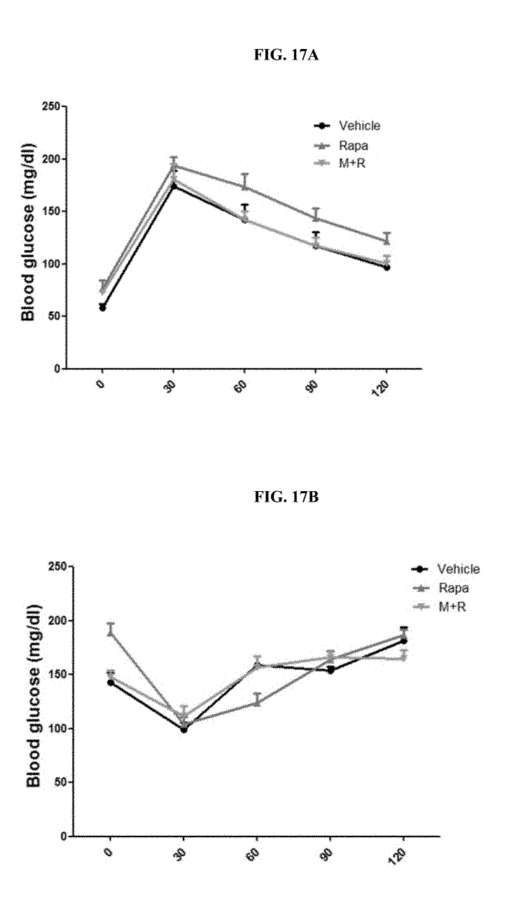

Mice were allowed to have arthritis induced by a high-fat diet and collagen in the same manner as in example <6-1>, and after 12 weeks, the mice in each group were tested for a glucose tolerance test and an insulin resistance test by intraperitoneal injection of glucose, and the change in blood glucose level over time was observed FIGS. 17A and 17B.

The intraperitoneal glucose tolerance test was performed by intraperitoneal injection of glucose at 1 g/kg of body weight after fasting for 12 hours. The insulin tolerance test was performed at 30 minutes interval after the subcutaneous injection of glucose at 1 U/kg of body weight.

As a result of the intraperitoneal glucose tolerance test (FIG. 17A), the blood glucose level was the highest in the rapamycin administered alone group (Rapa) among the three groups. The blood glucose level of the metformin co-administered group was maintained at a similar level to the control group (Vehicle), and was significantly lower compared with the rapamycin administered alone group.

As a result of the insulin tolerance test (FIG. 17B), the glucose level was the highest at the start point of measurement in the rapamycin administered alone group (Rapa) among the three groups. Thereafter, the blood glucose level was maintained at similar levels in the metformin co-administered group (Rapa+Met), the rapamycin administered alone group, and the control group (Vehicle).

<6-3> Blood Test of Arthritis Animal Models

The treatment effect of the rapamycin and metformin co-administration in the arthritis disease animal models was examined. In the mouse models with arthritis induced by a high-fat diet and collagen in the same manner as in example <6-1>, the effects between the administration of rapamycin alone and the co-administration of rapamycin and metformin were compared.

Seven-week-old DBA1/J mice were orally administered with rapamycin (1 mg/kg) alone or together with metformin (50 mg/kg) with arthritis induction stimulation and a high-fat diet for 12 weeks, and then sacrificed. Thereafter, the serum glucose, triglyceride, and free fatty acid levels were measured.

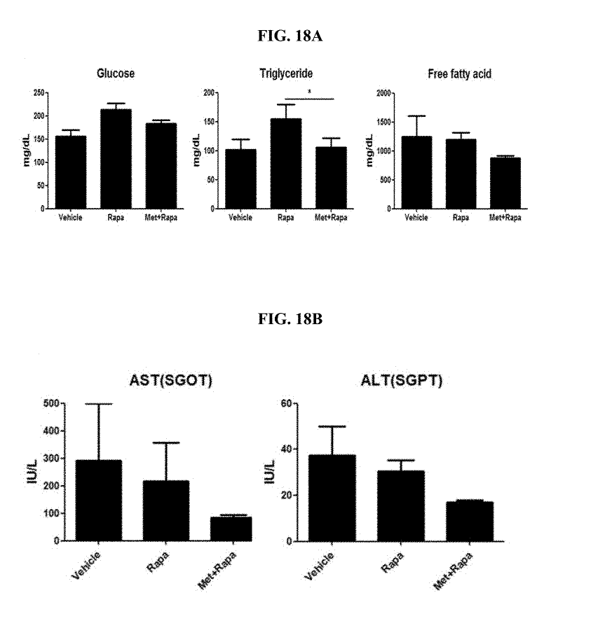

As shown in FIG. 18A, it could be confirmed that the levels of glucose, triglyceride, and free fatty acid were reduced in the metformin and rapamycin co-administered group compared with the rapamycin administered alone group.

In order to examine the effect of metformin and rapamycin co-administration on fatty liver symptoms in the arthritis animal models, the serum AST and ALT levels were measured. When the structures and functions of the cell membranes are destroyed, aspartate aminotransferase (AST) and alanine aminotransferase (ALT), which are widely distributed in the cytoplasm of hepatic cells, are released into the blood, and thus, the levels of AST and ALT in blood are frequently used as indicators of liver injury.

The AST and ALT activities were measured by using a quantitative kit reagent (Yeongdong Pharm, Korea). 1.0 mL of AST and ALT substrate solutions were warmed in a 37□ water bath for 2 minutes, then 0.2 mL of plasma was added thereto, followed by incubation in a 37□ water bath for 30 minutes. After 30 minutes, 1.0 mL of the color development reagent was added, and the mixture was left at room temperature for 20 minutes. Then, 10.0 mL of 0.4 N NaOH was added, and the absorbance was measured at 505 nm. The AST and ALT standards (2 mM pyruvate) were developed according to the concentration by the same method as above, followed by absorbance measurement. Then, the activity of each sample was calculated by extrapolation on the standard curve.

As shown in FIG. 18B, it could be confirmed that the levels of AST and ALT were significantly reduced in the metformin and rapamycin co-administered group compared with the rapamycin administered alone group.

### INDUSTRIAL APPLICABILITY

As set forth above, the composition of the present invention effectively alleviates the mitochondrial dysfunction induced by side effects of an existing immunosuppressant, and thus can be advantageously used in the improvement of the treatment effect of a disease in need of immunosuppression. Furthermore, another pharmaceutical composition or complex preparation of the present invention presents various methods for co-administration of an existing immunosuppressant and metformin, and thus reduces the mitochondrial dysfunction side effect of an existing immunosuppressant and maximizes the immunosuppressive or immunoregulatory effect, so that the pharmaceutical composition or complex preparation of the present invention is effective in the prevention or treatment of a rejection of organ transplantation, an autoimmune disease, or an inflammatory disease, and thus is highly industrially applicable.

1. A method for treating an immunosuppressant-induced mitochondrial disease in a subject in need thereof, the method comprising administering a subject in need thereof an effective amount of a composition comprising metformin or a pharmaceutically acceptable salt thereof as an active ingredient.

2. The method of claim 1, wherein the immunosuppressant-induced mitochondrial disease is caused by at least one mitochondrial dysfunction selected from the group consisting of mitochondrial respiration suppression, mitochondrial membrane potential reduction, and mitochondrial activity reduction.

3. The method of claim 1, wherein the immunosuppressant is a mammalian target of rapamycin (mTOR) inhibitor.

4. The method of claim 3, wherein the mTOR inhibitor is rapamycin or a derivative thereof.

5. The method of claim 4, wherein the derivative of rapamycin is selected from the group consisting of everolimus, temsirolimus, and deforolimus.

6. A method for treating an immune disease in a subject in need thereof, the method comprising administering a subject in need thereof an effective amount of a composition comprising an mTOR inhibitor and metformin or a pharmaceutically acceptable salt thereof as active ingredients.

7. The method of claim 6, wherein the mTOR inhibitor is rapamycin or a derivative thereof.

8. The method of claim 6, wherein the weight ratio of the mTOR inhibitor to metformin or the pharmaceutically acceptable salt thereof is in a range of 1:500 to 1:200,000.

9. The method of claim 6, wherein the immune disease is selected from the group consisting of an acute or chronic rejection of organ transplantation, an autoimmune disease, and an inflammatory disease.

10. A pharmaceutical complex preparation for treating an immune disease, the complex preparation being characterized in that: (a) the pharmaceutical complex preparation comprises an mTOR inhibitor and metformin or a pharmaceutically acceptable salt thereof at a weight ratio of 1:500 to 1:200,000; and
(b) the mTOR inhibitor and metformin or the pharmaceutically acceptable salt thereof are administered simultaneously, individually, or in a predetermined order.

11. The pharmaceutical complex preparation of claim 10, wherein the mTOR inhibitor is rapamycin or a derivative thereof.

12. The pharmaceutical complex preparation of claim 10, wherein the immune disease is selected from the group consisting of an acute or chronic rejection of organ transplantation, an autoimmune disease, and an inflammatory disease.

13-16. (canceled)
